(19) United States
(12) Patent Application Publication
(10) Pub. No.: US 2019/0017999 A1
(43) Pub. Date: Jan. 17, 2019

(54) IN VITRO SKIN IMMUNE SYSTEM SIMULATION SYSTEM

(71) Applicant: Seoul National University R&DB Foudation, Seoul (KR)

(72) Inventors: Noo Li Jeon, Seoul (KR); Min Hwan Chung, Seoul (KR); Su Jung Oh, Seoul (KR); Woo Hyun Park, Seongnam, Gyeonggi-do (KR); Hyun Jae Lee, Seongnam, Gyeonggi-do (KR); Hyun Yul Ryu, Seoul (KR); Su Dong Kim, Seoul (KR)

(21) Appl. No.: 15/757,564

(22) PCT Filed: Sep. 4, 2015
(86) PCT No.: PCT/KR2015/009379

§ 371 Date: Aug. 22, 2018

(51) Int. Cl. G01N 33/50; C12N 5/071; C12M 1/12; C12N 5/079; C12N 5/077; C12Q 1/02; B01L 3/00

(52) U.S. Cl. CPC G01N 33/5011; C12N 5/0691; C12M 25/04; C12N 5/0698; C12N 5/0622; B01L 2300/0877; C12Q 1/025; B01L 3/5027; C12N 2500/50; B01L 2300/0874; C12N 5/0652

(57) ABSTRACT

The present invention relates to a micro-fluid chip for blood vessel formation. The micro-fluid chip of the present invention is constituted by first to fifth channels arranged adjacent to one another on a substrate in sequence, and two or more micro-structures or micro-posts having a gap therebetween are disposed on the interface that each channel forms together with an adjacent channel while contacting the same. Each channel performs a fluidic interaction with a different channel through the gap formed by the micro-structures, and biochemical materials can move therethrough. The micro-fluid chip, according to the present invention, provides a micro-blood vessel having a flat and continuous blood vessel interface outside a body. Furthermore, cancer angiogenesis, cancer intravasation, and cancer extravasation can be modeled using the micro-fluid chip of the present invention. In addition, the micro-fluid chip of the present invention can be used to screen candidate anti-cancer drugs.

### TECHNICAL FIELD

The present invention relates to a microfluidic chip for co-culture of blood vessels or lymphatic vessels with pericytes. Moreover, the present invention relates to a system that mimics the skin's structural layers by co-culturing skin cells with microvessels, microlymphatic vessels and pericytes in vitro and that analyzes the mechanism of cancer metastasis, the in vivo mechanism of immune cells, or the like based on the co-culture results. In addition, the present invention relates to a method of co-culturing blood vessels and pericytes in vitro and screening a drug candidate based on the co-culture results.

### BACKGROUND ART

The formation and function of blood vessels is an important process that mediates many physiological and pathological processes. It is known that more than 70 diseases, including cancer development and metastasis, blindness caused by diabetes, senile macular degeneration, psoriasis and rheumatoid arthritis, are caused or exacerbated by abnormalities in blood vessel production and function. Therefore, studies on understanding the mechanisms of these processes and developing drugs and therapies for controlling these mechanisms can lead to overcoming various diseases. Fundamental studies and development of new drugs to overcome vascular diseases require the implementation of an experimental platform that mimics in vitro the formation and function of blood vessels and pathological processes. A system for culturing cells attached to a semi-permeable membrane (Gastroenterology 96(3), 736-749, 1989) can be regarded to be representative of conventional angiogenesis-related research methods or drug test methods, and requires less time and labor costs compared to experiments employing in vivo blood vessels, but has a disadvantage in that it cannot accurately mimic the biological characteristics of actual in vivo blood vessels, making it difficult to accurately predict the biological morphology of actual blood vessels.

The blood vessels in the human body have different characteristics depending on their positions. For example, cerebral and ocular blood vessels form blood-brain barriers (BBB) and blood-retinal barriers (BRB), which have significantly lower vascular permeability than elsewhere. It is believed that these barriers are a major factor that determines drug delivery characteristics, and that they are formed due to the influence of pericytes (which are representative cells surrounding blood vessels) and astrocytes on blood vessels. Thus, in vitro mimicking of this perivascular environment is a major factor in in vitro blood vessel mimicking models for new drug development.

The skin is composed of: an epidermal layer having keratinocytes; a dermal layer located under the epidermal layer and having microvessels and fibrous cells; and a subcutaneous fat layer having blood vessels and fat.

Microvessels extend from the blood vessels of the subcutaneous fat layer to immediately below the epidermal layer, and supply of nutrients and migration of immune cells occurs through the microvessels. Among skin cell lines, keratinocytes form the epidermis, and Langerhans cells are involved in immunity. Cells that immune cells are dendritic cells, and Langerhans cells are one type of dendritic cells. Dendritic cells extravasate from the blood vessel walls in vivo (extravasation) and undergo maturation while migrating to tissues. Exposure to UV rays for a long time in everyday life causes inflammation of the skin, because the skin tissue and the vascular system are connected organically. When keratinocytes in the skin tissue are exposed to UV rays, they induce an immune response to attract immune cells or secrete VEGF to induce new blood vessel formation. There has been a continuing need for a culture platform capable of analyzing the correlation between skin-derived cells and blood vessels and/or lymphatic vessels.

It is known that cancer cells continuously interact with surrounding blood vessels during cancer growth and metastasis (Bergers and Benjamin 2003; Carmeliet and Jain 2000).

As tumor cell clusters proliferate, they secrete factors that stimulate the surrounding blood vessels to go toward the clusters. This phenomenon is known as tumor angiogenesis. Tumor angiogenesis causes nutrients and oxygen to be supplied to tumor cells from blood vessels, causing tumors to grow into malignant tumors. A tumor cell composed of a fully grown ellipsoid passes through surrounding blood vessel cells, circulates in the blood, and passes through the secondary site in a region surrounding blood vessels (Sakai 2007). Extravasating tumor cells grow to form secondary tumor clusters. Most cancer deaths are caused by this recurrent cancer metastasis. Therefore, as a stage involved in the progression of cancer metastasis, studies mainly on tumor angiogenesis and migration through endothelial cells have been extensively conducted. In transwell experiments, cancer cells were grown in a lower chamber, and endothelial cells in extracellular matrices such as collagen or Martrigel were cultured in an upper chamber. Tube formation (Tsujii et al. 1998) and migration of endothelial cells (Abdollahi et al. 2005) can be observed in response to cancer-derived factors. In a model for migration of cancer cells through endothelial cells, the endothelial cells were cultured to confluence in a Petri dish or a porous membrane, and the permeation of the endothelial monolayer could be observed after several hours to several days of culture (Jin et al. 2012; Kramer and Nicolson 1979; Kusama et al. 2006; Lee et al. 2003; Roetger et al. 1998; Zabel et al. 2011).

Microfluidic technology has a variety of advantages over conventional methods with respect to precise regulation of microenvironmental factors, or high-resolution imaging control of cell migration or cell-cell interactions (Chung et al. 2010; Lee et al. 2014a). An angiogenic process was modeled by attaching endothelial cells to a hydrogel wall and co-culturing the endothelial cells with cancer cells to stimulate the endothelial cells (Kim et al. 2013). The disadvantage of this experiment is that a developmental process begins from endothelial cell clusters deficient in appropriate cell-cell junctions. Since angiogenesis occurs from fully established blood vessels near cancer clusters, this characteristic is not physiologically related to actual in vivo angiogenesis. In another study, vascular walls were modeled by culturing a monolayer of endothelial cells in a microfluidic channel (Jeon et al. 2013; Zervantonakis et al. 2012). Migration through endothelial cells can be modeled by introducing cancer cells into the system. However, this vascular model involves attaching a monolayer of endothelial cells onto a hydrogel without an angiogenic or vasculogenic process. Recently, in order to analyze the extravasation of cancer, a microfluidic model has been developed which generates a vascular network by an angiogenic process (Chen et al. 2013). However, this model did not allow the concentration gradient direction of chemokines to induce chemotactic migration of cancer cells, and the unpredictable geometry of the vascular network has increased the complexity of the experiment.

Accordingly, the present inventors have made efforts to solve the above-described problems occurring in the prior art, and as a result, have found that skin cells, skin blood vessels, skin lymphatic vessels and skin pericytes can successfully co-cultured by using a microfluidic chip of the present invention, thereby completing the present invention.

Using the microfluidic chip of the present invention, it is possible to obtain microvessels having a smooth and continuous boundary, formed by a natural angiogenic process. In addition, the obtained microvessels may be used to model cancer angiogenesis and intravasation. As described above, the microfluidic chip of the present invention can be widely used in the fields of basic cancer biology, drug candidate screening and the like.

### PRIOR ART DOCUMENTS

### Non-Patent Documents

Bergers and Benjamin 2003, Nature Reviews Cancer, 3(6), 401-410.

Carmeliet and Jain 2000, Nature, 407 (68010, 249-257.

Sahai E. 2007, Nature Reviews Cancer 7(10):737-749.

Tsujii et al. 1998, Cell 93(5):705-716.

Abdollahi et al. 2005, Clinical Cancer Research 11(17):6270-6279.

Jin et al. 2012, Molecular Cancer Research 10(8):1021-1031.

Kramer and Nicolson 1979, Proceedings of the National Academy of Sciences 76(11):5704-5708.

Kusama et al. 2006, International journal of oncology 29(1):217-223.

Lee et al. 2003, Journal of Biological Chemistry 278(7):5277-5284.

Roetger et al. 1998, The American journal of pathology 153(6):1797-1806.

Zabel et al. 2011, Mol Cancer 10(73):10.1158.

Annals of Biomedical Engineering 38(3):1164-1177.

Lee et al. 2014a, MRS Bulletin 39(01):51-59.

Kim et al. 2013, Lab Chip 13(8):1489-1500.

Jeon et al. 2013, Integrative Biology 5(10):1262-1271.

Zervantonakis et al. 2012, Proc Natl Acad Sci USA 109(34):13515-13520.

Chen et al. 2013, Integrative Biology 5(10):1262-1271.

Xia, Y. et al. 1998, Angew. Chem. Int. Ed. Engl. 37 (5): 551-575.

Lee et al. 2014b, Microvasc Res 91:90-98.

Flament et al. 2013, Magnetic Resonance in Medicine 69(1):179-187.

Yuan et al. 1996, Proceedings of the National Academy of Sciences 93(25):14765-14770.

Pechman et al. 2011, Journal of neuro-oncology 105(2):233-239.

Wu et al/2001, American Journal of Physiology-Cell Physiology 280(4):C814-C822.

Zen et al. 2008, PloS one 3(3):e1826.

(rett et al. 1989, The Journal of experimental medicine 169(6):1977-1991.

Burke-Gaffeyy and Keenan 1993, Immunopharmacology 25(1):1-9.

Horvath et al. 1988, Proceedings of the National Academy of Sciences 85(23):9219-9223.

Clinical cancer research 10(6):1901-1910.

Liang et al. 2007, Cancer letters 258(1):31-37.

Zervantonakis et al. 2012, Proc Natl Acad Sci USA 109(34):13515-13520.

### DISCLOSURE

### Technical Problem

Therefore, the present invention provides a method of cu-culturing blood vessels/lymphatic vessels and pericytes in a microfluidic chip.

The present invention also provides a microfluidic chip for generating tumor angiogenesis, and tumor angiogenesis generated in vitro by use of the microfluidic chip.

The present invention also provides a microfluidic chip for inducing tumor intravasation and extravasation, and tumor intravasation and extravasation induced in vitro by use of the microfluidic chip.

The present invention also provides a skin structural layer cultured in vitro by co-culturing blood vessels/lymphatic vessels and skin cells by use of the microfluidic chip according to the present invention. The skin structural layer may be provided as a skin tissue chip comprising blood vessels and lymphatic vessels. This skin structural layer may be used to mimic the skin immune system and to implement cell migration intravasation and extravasation) through blood vessels and lymphatic vessels. In addition, it may be used to study the change/aging of blood vessels caused by skin damage or to study the passage of immune cells through subcutaneous blood vessels/lymphatic vessels and the mechanism of differentiation of undifferentiated cells after passage through vascular membranes.

The present invention also provides a method of screening a drug candidate by use of the microfluidic chip according to the present invention.

The present invention also provides a biological tissue chip comprising blood vessels/lymphatic vessels and co-culture cells formed in the microfluidic chip of the present invention.

The present invention also provides a method of generating blood vessels/lymphatic vessels and cells, which interact with each other in vitro, by use of the biological tissue chip of the present invention.

### Technical Solution

To achieve the above objects, in one embodiment, the present invention provides a microfluidic chip comprising: at least one blood vessel channel; at least one cell channel; and at least one medium channel. In the microfluidic chip, the blood vessel channel, the cell channel and the medium channel are disposed adjacent and parallel to one another such that they may be in fluidic communication with one another; both sides or one side of the blood vessel channel is adjacent to the medium channel, both sides or one side of the cell channel is adjacent to the other side of the medium channel; at the interface between the channels adjacent to each other, two or more microstructures, microposts or columns, which have various sectional shapes, for example, a triangular or hexagonal shape, are disposed with a predetermined gap; the medium channel is connected with a medium reservoir such that they may be in fluidic communication with each other, and each of the blood vessel channel and the cell channel is connected with its inlet such that they may be in fluidic communication with each other.

In another embodiment of the present invention, there is provided a biological tissue chip configured such that blood vessels or lymphatic vessels and cells, co-cultured in vitro, interact with each other, the biological tissue chip comprising: at least one blood vessel channel and blood vessels or lymphatic vessels or a combination of blood vessels or lymphatic vessels, formed in the blood vessel channel; at least one cell channel and cells cultured in the cell channel; and at least one medium channel, wherein the blood vessel channel, the cell channel and the medium channel are disposed adjacent and parallel to one another such that they may be in fluidic communication with one another; both sides or one side of the blood vessel channel is adjacent to the medium channel, both sides or one side of the cell channel is adjacent to the other side of the medium channel, and two or more barrier structures or microstructures are disposed at the interface between adjacent two of the channels with a gap; the medium channel is connected with a medium reservoir such that they may be in fluidic communication with each other, and each of the blood vessel channel and the cell channel is connected with its inlet such that they may be in fluidic communication with each other; each of the channels allows the interaction between biochemical substances contained in the channels through the gap; blood vessels or lymphatic vessels are formed from angiogenic or lymphangiogenic cells in the blood vessel channel, and cells are cultured in the cell channel; and the cultured cells interact with the formed blood vessels or lymphatic vessels. In one embodiment, the biological tissue mimics the skin tissue comprising the subcutaneous fat layer, the dermal layer and the horny layer.

In another embodiment of the present invention, the microfluidic chip 10 comprises a first channel 110, a second channel 120, a third channel 130, a fourth channel 140, and a fifth channel 150, which are sequentially disposed over a substrate, wherein the first channel is adjacent to one side of the second channel, the other side of the second channel is disposed adjacent and parallel to one side of the third channel, the other side of the third channel is disposed adjacent and parallel to one side of the fourth channel, and a portion or all of the other side of the fourth channel is disposed adjacent and parallel to one side of one or more fifth channels; and when two or more fifth channels are provided, the fifth channel is divided into two cells 170 and 180 by a barrier 160 provided in direction perpendicular to its interface with the third channel, and the cells may comprise fifth channels 151 and 152, respectively. Two or more microstructures 610 at an interface formed between adjacent channels in the microfluidic chip 10 are disposed with a predetermined gap 620, and each of the channels allows the interaction between biochemical substances contained in the channels through the gap.

In still another embodiment of the present invention, the microfluidic chip 20 comprises: a first channel 210 configured to be in fluidic communication with a first medium reservoir 201; a second channel 220 configured to be in fluidic communication with a second medium reservoir 202 and disposed parallel to the first channel 210; a blood vessel channel 230 configured to be in fluidic communication with a blood vessel channel inlet 203 and disposed between the first channel 210 and the second channel 220 and adjacent to one side of the first channel 210 and the second channel 220; and a cell channel 240 configured to be in in fluidic communication with a cell channel inlet 204 and adjacent to the other side of the second channel 220 and disposed parallel to the second channel 220. The microfluidic chip 20 comprises two or more barrier structures 660 protruding from the blood vessel channel 230, wherein each of the barrier structures 660 comprises: a first barrier 601 disposed parallel to the first channel 210; a second barrier 602 formed to extend from the first barrier 601 to the second channel 220; a third barrier 603 formed to extend from the second barrier 602 and disposed parallel to the first barrier 601; and at least one protrusion 604 formed in a space surrounded by the first barrier 601, the second barrier 602 and the third barrier 603, wherein the interface between the channels allows the interaction between biochemical substances in the channels.

In still another embodiment of the present invention, a microfluidic channel 30 comprises: a first channel 310 configured to be in fluidic communication with a first medium reservoir 301; a second channel 320 configured to be in fluidic communication with a second medium reservoir 302 and disposed parallel to the first channel 310; a first blood vessel channel 330 configured to be in fluidic communication with a first blood vessel channel inlet 303 and adjacent to one side of the first channel 310; a second blood vessel channel 340 configured to be in fluidic communication with a second blood vessel channel inlet 304 and adjacent to the other side of the second channel 330; a first cell channel 350 configured to be in fluidic communication with a first channel inlet 305 and adjacent to the other side of the first channel 310 and disposed parallel to the first channel 310; and a second cell channel 360 configured to be in fluidic communication with a second cell channel inlet 306 and adjacent to the other side of the second channel 310 and disposed parallel to the second channel 320.

In the microfluidic chip of the present invention, the height and width of the channel are not particularly limited, and may be determined flexibly according to the type of material to be injected into the channel, and the purpose and conditions of the experiment. In one embodiment, the height of the channel 10 μm to 1000 μm, preferably 500 μm to 800 μm.

In still another embodiment of the present invention, there are provided microvessels produced using the microfluidic chip 10. Specifically, production of the microvessels is performed by supplying a mixture of fibroblasts (e.g., lung fibroblasts (LF)) and fibrin to the fifth channel 150 and supplying a mixture of endothelial cells and fibrin to the third channel 150. During formation of the microvessels, the first channel 110 and the second channel 120 are maintained in an empty state. The second channel 120 should contain only air during formation of the microvessels in order to inhibit the formation of concentration gradient of growth factors from the second channel 120 and to prevent endothelial cells from moving toward an empty channel or from forming sprouts. To form microvascular cells having a smoother and more continuous boundary, endothelial cells are injected at a concentration of 2 to 10×10⁶ HUVECs/m, preferably 4 to 8×10⁶ HUVECs/ml, most preferably 6×10⁶ HUVECs/ml. At a low HUVEC concentration of 3×10⁶ cells/ml (FIG. 2A), Endothelial cells form thicker blood vessels than in the lower region. Conversely, when the HUVEC concentration is excessively high (e.g., 9×10⁶ cells/ml; FIG. 2A), excessive endothelial cells are present in the blood vessel channel, thus interfering with migration of endothelial cells and formation of a smooth blood vessel boundary. Thus, the optimal HUVEC concentration is 6×10⁶ cells/ml. At the same time, medium is supplied from the fourth channel 140, and this medium acts to move the endothelial cells to the lower region and causes the microvessels to be closer to the fourth channel 140. During formation of microvessels according to the present invention, the endothelial cells existing in the gap between microposts tend to move toward a lower region having the medium channel, and for this reason, a smooth vascular boundary is formed (FIG. 2C).

In still another embodiment, the present invention provides tumor angiogenesis using the microfluidic chip 10. Specifically, microvessels are formed according to the present invention, and then a mixture of a cell line having angiogenic ability (e.g., the U87MG cell line) and fibrin is injected into the first channel, followed by injection of fibrin into the second channel.

In still another embodiment, the present invention provides a platform for mimicking the immune function of a skin structural layer using the microfluidic chip 10.

In still another embodiment, the present invention provides a method of screening an anticancer drug candidate. Specifically, the screening method of the present invention comprises: treating a cell line, which has the angiogenic ability to induce tumor angiogenesis, with a sample; and determining that when tumor angiogenesis is not generated, the sample is an anticancer drug candidate.

In still another embodiment, the present invention provides tumor intravasation using the microfluidic chip 10. Specifically, after tumor angiogenesis is generated according to the present invention, cancer cells (e.g., MDA-MB-231 cells) are attached to a fibrin gel in the second channel 120, and a medium for cancer cell growth is supplied to the first channel 110. Next, when growth factor-free medium (EBM) is supplied to the first channel 110, cancer intravasation is induced by the chemotactic migration of cancer cells to the microvascular wall.

In another embodiment of the present invention, the endothelial cells have proper cell-cell junctions and shows an elongated shape as in vivo, and the medium can be perfused through the vascular lumen, and it has two three-dimensional vascular structures opened toward the fourth channel 140. In addition, the present invention makes it possible to successfully model cancer angiogenesis and the inhibition of the angiogenic pathway by treatment with an anti-vascular endothelial growth factor (anti-VEGF; bevacizumab). In addition, one embodiment of the present invention shows the regulation of tumor intravasation rate by treatment with tumor necrosis factor-alpha (TNF-a).

In the present invention, extracellular matrix or extracellular matrix/angiogenic cells are filled in the blood vessel channel is filled with, thereby forming a three-dimensional angiogenic region. The angiogenic region provides a space in which angiogenic cells can three-dimensionally proliferate and differentiate to form blood vessels. At the interface between the blood vessel channel and the medium channel, specific barrier structures 660 or other microstructures 610 are arranged with a gap. When extracellular matrix or extracellular matrix/angiogenic cells are injected into the blood vessel channel, the extracellular matrix or extracellular matrix/angiogenic cells form an angiogenic region due to surface tension without escaping through the microstructures. On the other hand, since various biochemical substances contained in the channels can move through the gap between the microstructures, various angiogenic factors, nutrients, etc. contained in other channels can be supplied to the blood vessel channel and/or the cell channel.

In addition, blood vessels generated in the angiogenic region may extend toward the gap formed between the plurality of microstructures and communicate with a channel adjacent to the blood vessel channel, thereby forming an inlet and outlet for blood vessels. Thus, it is possible to observe and image the reaction of blood vessels in real time by transferring various biochemical and biophysical substances and signals directly into the vascular lumens through the channel communicated with the gap between the plurality of microstructures. Accordingly, it is possible to control the number of inlets and outlets of the vascular network to be generated, by changing the arrangement, number, size, etc. of the microstructures of the vascular channel, in particular, the barrier structures 660. It is obvious that parameters such as the shape of the microstructures are not limited to the embodiment shown in the drawings but can be appropriately adjusted according to the purpose and configuration of the experiment.

The liquid extracellular matrix or extracellular matrix/angiogenic cells prior to curing injected into the vessel channel form a meniscus by capillary action and surface tension between the plurality of microstructures. Below a threshold pressure level, the meniscus does not advance to an adjacent channel due to surface tension. The range of pressure, in which the meniscus can stop between the plurality of microstructures without advancing to the side channel adjacent thereto, is influenced by the spacing between and height of the microstructures. Thus, the threshold pressure level can be adjusted by optimizing these two parameters (the spacing between the microstructures and the height of the microstructure). For a specific method of adjusting the threshold pressure level, reference may be made to the content disclosed in Carlos P. Huang et al. (Engineering microscale cellular niches for three-dimensional multicellular co-cultures, Lab Chip, 2009, 9, 1740-1748).

By adjusting the spacing between the microstructures and the height of the microstructures, the meniscus can be effectively stopped between the structures when extracellular matrix or extracellular matrix/angiogenic cells are injected into the vessel channel, thereby precisely controlling the filling of the extracellular matrix between the channels that are not completely physically separated from each other. This is also true when extracellular matrix or extracellular matrix/co-culture cells are filled in the cell channel.

In one embodiment of the present invention, the number of the barrier structures 660 in the blood vessel channel is 1 to 15, preferably 5 to 8. In another embodiment, one protrusion 604 is preferably included per barrier structure 660. The shape of the protruding portion 604 is preferably configured such that the length of one side in the direction of the blood vessel channel inlet is shorter than that of the other side in the opposite direction, that is, the length of the other side in the direction of an empty channel, if any. In another embodiment of the present invention, the second barrier 602 is preferably formed to extend perpendicularly from the first barrier 601, and the third barrier 603 may include a bent portion 605 in the direction of a space surrounded by the first barrier 601, the second barrier 602 and the third barrier 603. For example, the bent portion 605 may be bent toward the connection between the first barrier 601 and the second barrier 602. In another embodiment, when an empty channel is present, two microstructures are preferably disposed between the blood vessel channel and the empty channel. The microstructures may have the same shape as that of the protrusion 604 in the blood vessel channel.

Extracellular matrix or extracellular/co-culture cells are filled in the cell channel, thereby forming a three-dimensional cell culture region. At the interface between the cell channel and the medium channel, a plurality of microstructures may also be arranged with a gap. In this case, when extracellular matrix or extracellular/co-culture cells are injected into the cell channel, the extracellular matrix or extracellular/co-culture cells form a cell culture region in the cell channel due to surface tension without escaping through the microstructures. The cell culture region may provide a space in which the co-culture cells may be cultured three-dimensionally. This three-dimensional multicellular co-culture can physiologically mimic in vivo environments and can promote the production and secretion of various signal substances from the co-culture cells. Meanwhile, since various kinds of biochemical substances such as angiogenic factors contained in the medium channel and various signal substances generated in the cell channel can move through a gap between the plurality of microstructures, the active interaction of biochemical substances between the cell channel and other channel adjacent thereto may occur.

The medium channel that provides medium provides a passage allowing the flow of a fluid, wherein the fluid may include cell culture medium, various angiogenic factors, and the like. One of the medium channels is placed in contact with one side of the blood vessel channel and the cell channel, and the other medium channel is placed adjacent to the other side of the blood vessel channel, and thus these medium channels supply cell culture medium, angiogenic factors and the like to the blood vessel channel and the cell channel. This allows long-term cell culture of angiogenic cells and co-culture cells, and provides a passage through which the two cell groups (angiogenic cells and co-culture cells) in the blood vessel channel and the cell channel

allows passage of both vascular channels and two cell groups (angiogenic and co-culture cells) in the cell channel can interact through a paracrine mechanism. Paracrine interaction between angiogenic cells and co-culture cells promotes the morphogenesis of the angiogenic cells through the vascular network, and also has an important effect on the expression of specific properties and function of formed blood vessels.

In addition, one of the medium channels physically separates between angiogenic cells and co-culture cells, thereby making it easy to independently observe and analyze the angiogenic cells and the co-culture cells. In particular, it provides a space allowing various biochemical and biophysical materials and signals to be injected into blood vessels communicating with the medium channel through the gap between the plurality of microstructures.

The microfluidic chip according to the present invention can be fabricated by, for example, a lithography method, a molding method or the like, but is not limited thereto. In one embodiment, photoresist is deposited on a silicon wafer to a thickness of 50 to 100 μm, and ultraviolet light is irradiated thereto through a pattern formed on a light transmissive film to cure the photoresist, thereby forming a structure. Polydimethylsiloxane (PDMS) may be poured and cured on the formed structure which is then bonded to thin glass, thereby fabricating an angiogenic device.

According to the present invention, co-cultivation of angiogenic cells and pericytes is possible, and the two cell groups (angiogenic and co-culture cells) in the blood vessel channel and the cell channel can interact through a paracrine mechanism. The paracrine interaction between angiogenic cells and co-culture cells promotes the morphogenesis of the angiogenic cells through the vascular network, and also has an important effect on the expression of specific properties and function of formed blood vessels. In addition, one of the medium channels physically separates between angiogenic cells and co-culture cells, thereby making it easy to independently observe and analyze the angiogenic cells and the co-culture cells. In particular, it provides a space allowing various biochemical and biophysical materials and signals to be injected into blood vessels communicating with the medium channel through the gap between the plurality of microstructures. This allows to mimic the interaction between various biochemical/biophysical substances and blood vessels, and to observe the response of blood vessels to various stimuli and to image and quantify this response in real time.

In another aspect, the present invention is directed to an in vitro angiogenesis method comprising a step of sequentially or simultaneously injecting one or more, selected from the group consisting of angiogenic cells, extracellular matrices, cell culture media, angiogenic factors and co-culture cells, into one or more independent channels of the microfluidic chip according to the present invention, culturing angiogenic cells, and inducing blood vessel formation. The blood vessel formation in the present invention may be an angiogenesis process and/or a vasculogenesis process.

Injection of extracellular matrix, cells, cell culture medium, various angiogenic factors, etc. into each channel, does not require a separate external laboratory device, and may be simply performed by pipetting.

In addition, angiogenic cells and co-cultured cells may be injected as a mixture with extracellular matrix, but only the extracellular matrix may be injected depending on the purpose of the experiment. The extracellular matrix injected into each channel is cured by a rise in temperature, chemical action, light irradiation, etc. depending on the type of extracellular matrix injected.

In another embodiment of the present invention, there is provided a method for generation of blood vessels/lymphatic vessels and cells, which interact with each other in vitro, the method comprising: sequentially or simultaneously injecting one or more, selected from the group consisting of angiogenic cells, lymphangiogenic cells, extracellular matrices, cell culture media, angiogenic factors, lymphangiogenic factors and co-culture cells, into one or more independent channels of the microfluidic chip according to the present invention; culturing angiogenic cells; inducing blood vessel formation; and culturing co-culture cells.

In another embodiment of the present invention, there is provided a method for generation of blood vessels or lymphatic vessels and cells, which interact with each other in vitro, the method comprising the steps of: (a) injecting extracellular matrix and angiogenic or lymphangiogenic cells into the blood vessel channel of the microfluidic chip according to the present invention; (b) injecting extracellular matrix or extracellular matrix and co-culture cells into the cell channel; and (c) injecting cell culture medium, angiogenic or lymphangiogenic factor, or a combination of cell culture medium and angiogenic or lymphangiogenic factor into the medium channel, inducing blood vessel or lymphatic vessel formation in the blood vessel channel, and culturing the co-culture cells in the cell channel.

In still another embodiment of the present invention, there is provided a method for generation of blood vessels or lymphatic vessels and cells, which interact with each other in vitro, the method comprising the steps of: (a) injecting extracellular matrix or extracellular and co-culture cells into the blood vessel channel of the microfluidic chip according to the present invention, and forming a cell adhesion surface for cell adhesion at the interface between the blood vessel channel and the medium channel; (b) injecting angiogenic cells into the medium channel, and allowing the angiogenic cells to adhere to the cell adhesion surface; (c) injecting extracellular matrix or extracellular matrix and co-culture cells into the cell channel; and (d) injecting cell culture medium, angiogenic factors, or cell culture medium and angiogenic factors into the medium channel, culturing in the angiogenic cells in the blood vessel channel, and inducing blood vessel formation.

In still another embodiment of the present invention, there is provided a system capable of mimicking the skin immune system by culturing immune cells in the cell channel of the microfluidic chip according to the present invention.

In the present invention, when angiogenic cells are adhered to the extracellular matrix or extracellular matrix and co-culture cells on the cell adhesion surface, the angiogenic cells contained in the cell culture medium are injected into the channel, and the angiogenic device is tilted by an angle of about 90° for about 10 to 40 minutes, so that the angiogenic cells can be adhered to the intended location.

In the present invention, in order to promote or control the culture and morphogenesis of angiogenic cells depending on the type of co-culture cells and the purpose of the experiment, angiogenic factors or other factors that influence cell responses may be added to extracellular matrix or cell culture medium. Angiogenic sprouts form blood vessels that can grow and spread from the angiogenic region having angiogenic cells attached thereto. In order to provide a directional concentration gradient of angiogenic factors that induce angiogenesis from angiogenic cells, the location of adhesion of angiogenic cells, the injection of co-culture cells, and the introduction of angiogenic factors into the medium channel, may be suitably selected depending on the purpose of the experiment.

In the present invention, the term “angiogenic cells” refers to cells that form blood vessels by interaction with an angiogenic factor, an angiogenic inducer contained in cell culture medium, co-culture cells, and the like. These angiogenic cells can form blood vessels through vasculogenesis and/or angiogenesis.

In the present invention, the angiogenic cells are not particularly limited and may be appropriately selected depending on the purpose of the experiment, but may preferably be one or more selected from the group consisting of endothelial cells, epithelial cells, cancer cells, stem cells, stem cell-derived cells, and endothelial progenitor cells. For example, the angiogenic cells may be endothelial cells derived from various tissues of the body, for example, human umbilical vein endothelial cells (HUVEC), human microvascular endothelial cells, human brain microvascular endothelial cells, human lymphatic endothelial cells, and the like. In addition, cancer cells may also be used to study cancer growth and metastasis mechanisms, etc. The culture cells that are used in the present invention may be vascular endothelial cells derived from various species other than humans, for example, porcine, murine or bovine species.

In addition, the angiogenic cells may be mutated cells, transfected cells, or mutated and transfected cells.

In the present invention, the extracellular matrix is not particularly limited, but may be one or more selected from then group consisting of collagen gel, fibrin gel, Matrigel, self-assembled peptide gel, polyethylene glycol gel, and alginate gel.

In the present invention, the co-culture cell may be cells that secretes biochemical substances necessary for angiogenesis, such as an angiogenic inducer and the like, through interaction with angiogenic cells. In the present invention, the co-culture cells are not particularly limited, but may be one or more selected from the group consisting of cells of the immune system, astrocytes, glial cells, mesothelial cells, fibroblasts, smooth muscle cells, cancer cells, pericytes, neuroglial cells, stem cells, stem cell-derived cells, and cells that interact with vascular endothelium. For example, when the blood vessels to be generated are brain blood vessels, the co-culture cells are preferably astrocytes, glial cells, mesothelial cells, or fibroblasts, and when the blood vessels to be generated are blood vessels other than brain blood vessels, the co-culture cells are preferably fibroblasts or smooth muscle cells. In addition, cancer cells may also be used as co-culture cells to study the relationship between cancer and angiogenesis. In addition, cells of the immune system may include T cells, B cells, macrophages, NK (natural killer) cells, or dendritic cells. In the present invention, co-culturing of cells of the immune system can complete an in vitro system that mimics the skin immune system. The type and combination of cells to be cultured and the culture method may be selected according to the purpose of the experiment.

In the in vitro angiogenesis method of the present invention, the co-culture cells may be mutated cells, transfected cells, or mutated and transfected cells.

In the in vitro angiogenesis method of the present invention, in order to quantitatively measure angiogenesis and functional effect and efficacy or to screen a new drug having the property of promoting or inhibiting angiogenesis, the extracellular matrix or the cell culture medium may include one or more selected from the group consisting of drugs, soluble factors, insoluble factors, biomolecules, proteins, nanomaterials, and siRNA (small interfering RNA).

In the in vitro angiogenesis method of the present invention, the angiogenic factors are not particularly limited, but may be vascular endothelial growth factor (VEGF), epidermal growth factor (EGF), and the like.

As used herein, the term “microstructure” is a micro-sized structure and refers to a collection of the microstructures 610, the gap 620, and channels and structures defined by them. The sectional shape of the microstructures 610 formed at the interface between the channels may be circular, triangular, or hexagonal, but is not limited thereto, and may also be a micropost or column shape. In one embodiment, the size (width) of the gap formed between the microstructures is 10 μm to 100 μm, preferably 50 μm to 90 μm, and more preferably 60 μm to 80 μm.

In the microfluidic chip 10 of the present invention, the first channel 110 is an upper channel; the second channel 120 is a bridge channel; the third channel 130 is a blood vessel channel; the fourth channel 140 is a medium channel; and the fifth channel 150 is an LF channel; and these terms have the same meaning as above.

As used herein, the term “empty channel” means a channel that allows the presence of gas therein but does not allow the presence of a liquid and a solid therein.

In the present invention, the term “blood vessels/lymphatic vessels” means “blood vessels or lymphatic vessels, and blood vessels or lymphatic vessels”, but it should be understood that it includes lymphatic vessels or blood vessels, even blood vessels or lymphatic vessels are used alone.

As used herein, the term “biochemical substances” is understood to include various substances, including cells, proteins, peptides, amino acids, cofactors, neurotransmitters, antioxidants, cofactors, lipids, carbohydrates, hormones, antibodies, antigens, or the like, which are required for biological functions and activities in the biochemistry field. The term is also understood to include common chemical compounds.

As used herein, the term “biological tissue chip” means a chip capable of mimicking in vivo tissue, which includes blood vessels/lymphatic vessels formed in the microfluidic chip of the present invention, as well as cells co-cultured therewith.

### Advantageous Effects

Using the microfluidic chip of the present invention, it is possible to obtain microvessels having a smooth and continuous boundary, formed by a natural angiogenic process. In addition, the present invention makes it possible to mimic a perivascular environment, which provides a basic environment for research on brain-blood vessel barriers (BBB), brain-retinal barriers (BRB), cancer angiogenesis and cancer intravasation, immune cells, skin cells, and the like. Thus, the present invention provides a more substantial means for forming vascular structures, which may be applied to various experimental models, including extravasation models of extracellular efflux of cancer cells or leukocytes, and mechanotransduction experiments on fluid movement through the vascular lumen. In addition, the present invention makes it possible to mimic experiments on the passage of immune cells through subcutaneous blood vessels, as well as differentiation processes after the passage of undifferentiated cells through vascular membranes. The microfluidic chip of the present invention may be widely used to screen drug candidates.

### DESCRIPTION OF DRAWINGS

FIG. 1 shows the structure of a microfluidic chip of the present invention and a design of experimental procedures. Specifically, FIG. 1A is a schematic view showing the structure of the microfluidic chip of the present invention.

FIG. 1B schematically shows microvessels generated according to the present invention.

FIG. 1C schematically shows cancer angiogenesis generated according to the present invention.

FIG. 1D schematically shows cancer intravasation generated according to the present invention.

FIG. 2 shows the results of optimizing the HUVEC concentration for generating smooth and continuous vascular walls.

FIG. 2A is a confocal micrograph of microvessels generated depending on the initial HUVEC concentration (7 days after HUVEC inoculation). When the HUVEC concentration was 6×10⁶ cells/ml, smooth and continuous vascular walls were formed.

FIG. 2B shows the success rate of formation of smooth microvascular walls. When the HUVEC concentration was 6×10⁶ cells/ml, the success rate of formation of smooth microvascular walls was the highest.

FIG. 2C depicts time-lapse micrographs of microvascular formation at a HUVEC concentration of 6×10⁶ cells/ml.

FIG. 3 depicts fluorescence micrographs of fully developed microvessels. Specifically, FIG. 3A shows lumens, formed in microvessels, through a three-dimensional (3D) projection and a cross-sectional image of the microvessels.

FIG. 3B is a micrograph showing before and after FITC-dextran solution is injected into microvessels.

FIGS. 3C and 3D show microvessels connected to one another. Smooth and clear lines of claudin-5 and ZO-1 suggest that proper connections have been formed. On day 2, dispersed HUVEC cells were elongated and began to differentiate into tubular forms. On day 4, HUVEC cells began to fuse together to form the lumen (interior space). On day 7, HUVEC cells were fully fused to form microvessels having substantial lumens and smooth vessel walls.

FIG. 4 depicts micrographs showing the results of a cancer angiogenesis experiment and shows the results of quantification. Specifically, FIG. 4A compares microscopic images of microvessel walls before and 3 days after injection of cancer cells. Microvascular sprouts induced by cancer were greatly reduced by treatment with bevacizumab.

FIGS. 4B and 4C show the results of quantifying the number and coverage area of sprouts under various conditions. Angiogenic sprouts from microvascular walls were formed toward the upper channel and promoted by the secretion of cancer cells. Treatment with bevacizumab significantly reduced the coverage area and number of sprouts, indicating the anti-angiogenic potential of bevacizumab in cancer treatment (***p<0.0005). The error bars represent SEM.

FIG. 5 depicts micrographs showing the results of a cancer intravasation experiment and shows the results of quantification. Specifically, FIG. 5A shows a three-dimensional micrograph of cancer cells that migrated through the microvascular walls (red: CD31, green: MDA-MB-231, blue: nucleus). Fluorescence and DIC micrographs show that cancer cells have penetrated into the microvascular walls.

FIG. 5B is a micrograph of VE-cadherin expressed on the cell-cell junctions in microvessels. Compared with microvessels (left) under normal conditions, VE-cadherin in TNF-α-treated microvascular exhibits a disrupted and pleated form (right), suggesting that the junctions were disrupted by the effect of TNF-α. Compared with a control experiment, TNF-a-treated microvessels show a very high rate of cancer intravasation, demonstrating the effect of TNF-a on the connection between microvessels and cancer intravasation (*p<0.0005 compared to control). The error bars represent SEM.

FIGS. 6, 7 and 8 schematically illustrate several forms of use of FIG. 1A.

FIGS. 9A, 9B and 9C illustrate a platform that mimics the immune function of the skin's structural layer according to the present invention.

FIG. 10 shows the structure of a microfluidic chip of the present invention.

FIG. 11A shows the structure of a microfluidic chip of the present invention, and FIGS. 11B and 11C show the result of co-culturing pericytes by use of the microfluidic chip.

FIGS. 12A to 12F show the results of analyzing the morphological characteristics of blood vessels generated by co-culturing pericytes by use of the microfluidic chip of the present invention.

FIG. 13 shows the results of observing the responses of blood vessels and pericytes to VEGF-A, TNF-a, and IL-1a, which are typical factors that frequently occur in the peritumoral environment and inflammatory conditions.

FIGS. 14 and 15 show the structure of a microfluidic chip of the present invention and two methods of co-culturing vascular cells and their peripheral cells.

FIGS. 16A and 16B shows the results of observing blood-astrocytes co-cultured using the microfluidic chip of the present invention.

FIGS. 17A and 17B show the results of observing blood vessels and lymphatic vessels cultured using the microfluidic chip of the present invention.

FIG. 18 shows a skin's structural layer mimicked using the microfluidic chip of the present invention.

FIG. 19 shows the structure of a microfluidic chip of the present invention.

FIG. 20 shows the results of observing the passage of immune cells and cancer cells through subcutaneous blood vessels by use of the microfluidic chip of the present invention.

### BEST MODE

Hereinafter, the constituent elements and technical features of the present invention will be described with reference to examples below. However, these examples are only to illustrate the present invention, and the scope of the present invention is not limited by these examples.

### Experimental Materials

Cell Culture, Immunostaining and Reagents

Human umbilical vein endothelial cells (HUVEC, Lonza) were cultured in endothelial cell growth medium (EGM-2, Lonza). Normal human lung fibroblasts (LF, Lonza) were cultured in fibroblast growth medium (FGM-2, Lonza). Human glioblastoma cells (U87MG, ATCC, Virginia) were cultured in DMEM medium supplemented with 10% fetal bovine serum (FBS), penicillin (100 U/ml) and streptomycin (100 U/ml). MDA-MA-231 cells were purchased from the ATCC (Manassas, Va.). MDA-MB-231 cells were transfected with a pEGFP plasmid, and cells were selected using 1 mg/ml G418 (A.G. scientific, Inc.). MDA-MA-231 GFP cells obtained from monoclones were incubated in RPMI1640 (WELGENE, Korea) supplemented with 10% fetal bovine serum, 1% penicillin/streptomycin (Gibco, BRL) and 250 μg/ml G418 (A.G. scientific, Inc.). All cells were incubated in a humidified incubator at 37° C. under 5% CO2. For immunostaining, endothelial cells were imaged using mouse monoclonal antibodies specific for human ZO-1 (Alexa Fluor594, clone ZO1-1A12, molecular probe), CD31 (AlexaFluor1647, clone WM59, Biolegends), VE-cadherin (eBioscience) and claudin-5 (Invitrogen), and the nucleus was stained with Hoechst 33342 (molecular probe). Bevacizumab (Avastin, Genentech) was diluted to 500 μg/ml, and microvessels introduced with cancer were treated with the dilution in cancer angiogenesis experiments. Recombinant human TNF-alpha (PetroTech) was diluted to 5 ng/ml, and microvessels were treated with the dilution at 24 hours before introduction of cancer.

Fabrication of Microfluidic Chip

A master mold was made by casting photoresist onto a silicon wafer. A 80-μm thickness mold was made according to the standard photolithography protocol (Xia, Y. et al. 1998) for SU-8100 (Microchem, US) photoresist. PDMS (Dow Corning, US) was poured onto the prepared master mold and cured in a dry oven at 80° C. PDMS and a cleaned coverslip were bonded together by plasma treatment (Femto Science, KR). To make the surface hydrophobic, the bonded device was kept in an oven at 80° C. for 48 hours or more.

Hydrogel and Cell Loading

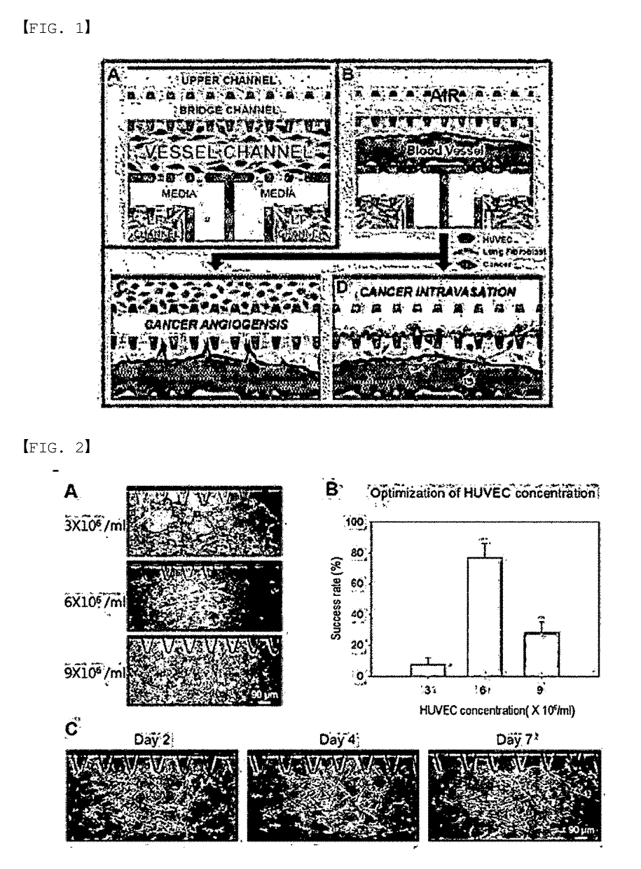

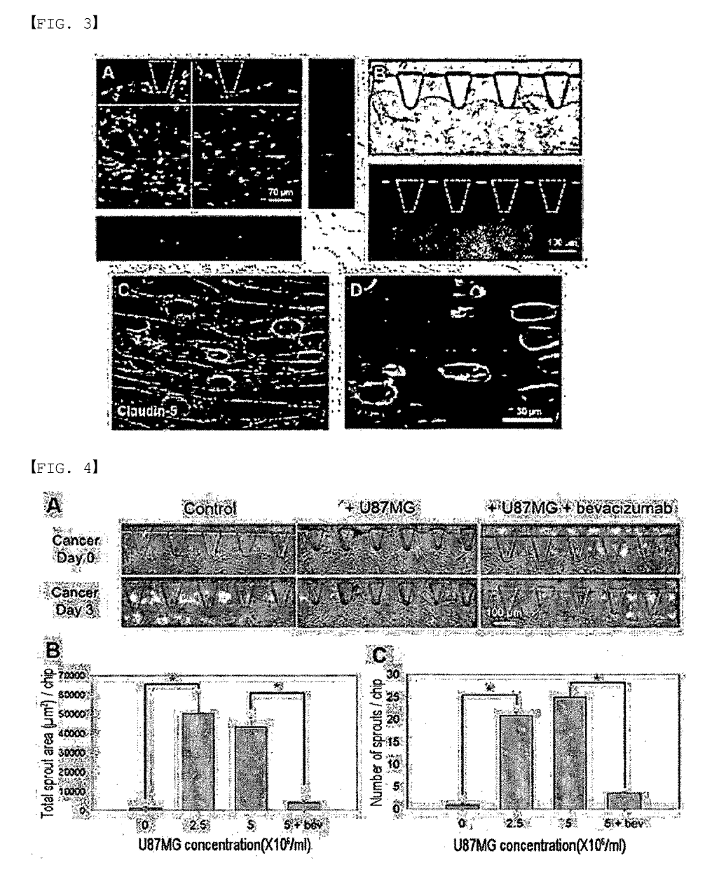

HUVECs (used at 6×10⁶ cells/ml in most experiments, and used at 3 or 9×10⁶ cells/ml in some experiments) and LFs (7×10⁶/ml) were mixed with a fibrinogen solution (2.5 mg/ml fibrinogen, 0.15 U/ml aprotinin and 0.5 U/ml thrombin) and injected into the third channel 130 (blood vessel channel) and the fifth channel 150, 151 or 152 (LF channel), respectively (FIG. 1A). For fibrin polymerization, incubation was performed for 2 minutes, after which EGM-2 medium was filled in the medium channel. The device was incubated for 7-8 days, thereby forming fully lumenized microvessels having open ends at each medium channel (FIG. 1B). After vascular maturation, U87MG and MDA-MA-231 cells were harvested from tissue culture dishes. For cancer angiogenesis, the U87MG cells treated with fibrinogen solution was injected into the first channel 110 (upper channel), and the second channel 120 (bridge channel) was filled with fibrinogen solution. For intravasation of cancer cells, MDA-MB-231 cells (1×10⁶ cells/ml) together with medium were injected into the second channel 120, and adhered to the fibrin wall between the second channel 120 and the third channel 130 (blood vessel channel) by tilting for 40 minutes. In order to induce chemotactic migration of cancer cells, EBM-2 (medium supplemented with no additional growth factor) was filled in the second channel 120 and the first channel 110, and EGM-2 medium was filled in the fourth channel 140.

Microscopy

For microvascular DIC (Differential Interference Contrast Microscope) imaging, the Nikon AE31 microscope was used. For 3D z-stack and cross-sectional imaging, stained samples were imaged using a confocal microscope (Olympus FV1000). Confocal images were analyzed using IMARIS software (Bitplane, Switzerland). For fluorescence imaging, FITC-dextran-injected samples were imaged using the IX81 inverted microscope (Olympus).

Data Analysis

To quantify the success rate of the smooth and continuous boundary of microvessels, the length of the vessel boundary was measured, and compared with the straight-line length of the blood vessel channel along the microvessel by use of image J. When the boundary length value was within ±10% of the linear length of the vascular channel, the chip was regarded as success. All microvessels having disconnected blood vessel walls were regarded as failure. Calculation of the permeability coefficient was performed using the method disclosed in the prior art (Non-Patent Document 19: Lee et al. 2014b). Specifically, FITC-dextran solution was introduced into microvessels and fluorescently imaged in every 15 seconds using multi-stage time-lapse mode in Metamorph. The acquired time-lapse image was analyzed using Image J, and the permeability coefficient was calculated using Equation 1 below:

P=1/lw×(dl/dt)/li

where lw is the length of the vessel wall that separates between perivascular region and microvessel region, li is the mean intensity in the microvessel region, I is the total intensity in the perivascular region.

For the quantification of cancer angiogenesis, sprout area and the number of sprouts were manually quantified by using Image J. For quantification of cancer intravasation, the microvessels were stained with CD31 and fluorescence imaged, and cancer cells at the apical side of the microvessel were manually counted using the IMARIS software.

### EXAMPLE

### Example 1: Fabrication of Microfluidic Chip

In previous works, the present inventors have described in detail the formation of a perfusable microvessel network in a microfluidic device using a co-culture system of HUVEC and LF. The HUVEC sprouts were stimulated by LF, which opened their lumens to both sides of the channel, allowing fluid passage through the vessels. However, the structure of microvessel in the previous model was unpredictable, and other cell types could not be introduced into the perivascular region, as the perivascular regions were filled with gel or PDMS (polydimethylsiloxane) wall. Therefore, the present inventors generated a microvessel with more predictable geometric characteristics and with perivascular regions that could be filled with other cell types after the generation of the microvessels with vasculogenic process. However, in this Example, the present inventors positioned two openings of the third channel 130 (blood vessel channel) on the same side (lower side), and interfaced the upper portion of the third channel 130 (blood vessel channel) with an empty channel (FIG. 1A). This generates a microvessel having two openings on the same side, while the other side of the microvessel is interfaced with the second channel 120, which is named the “bridge channel” in FIG. 1A.

The first channel 110 (upper channel) and the second channel 120 (bridge channel) were empty during microvessel growth. The interface with the empty channel prevented HUVECs in the upper region from migrating or generating sprouts toward the upper direction of the third channel 130 (blood vessel channel), and resulted in the generation of a smooth vessel wall, parallel to the interface.

FIGS. 1B to 1D show a schematic view of the generation of a microvessel, and the performance of the cancer angiogenesis and intravasation assays using the microfluidic chip 10. The present inventors first injected an LF-fibrin mixture into the fifth LF channel 150, 151 or 152 (LF channel), and a HUVEC-fibrin mixture into the third channel (blood vessel channel) to generate the microvessel. Medium was added to connect the cell-loaded channels. A microvessel with two openings toward the fourth channel 140 (medium channel) was generated after 7-8 days of incubation (FIG. 1B). Next, cancer angiogenesis was modeled using the U87MG cell line (obtained from the Korean Cell Line Bank) known to have high angiogenic potential as a cancer sprout inducer from the microvessel. The present inventors injected an U87MG-fibrin mixture into the first channel 110 (upper channel), followed by fibrin injection into the second channel 120 (bridge channel) (FIG. 1). The present inventors observed the formation of cancer sprouts from the pre-existing microvessels toward the cancer site, which was promoted by the secretion of pro-angiogenic factors from U87MG cells. The present inventors attached MDA-MB-231 cells (obtained from the Korean Cell Line Bank) to the fibrin gel exposed to the second channel 120 (bridge channel), and supplied medium to the first channel 110 (upper channel) for the cancer intravasation assay (FIG. 1D). Growth factor-free medium (EBM-2) was supplied to the first channel 110 (upper channel) to induce chemotactic migration of the cancer cells toward the microvessel wall. After 2-3 days of incubation, intravasated cancer cells were observed inside the microvessels, indicating successful modeling of the cancer intravasation process.

### Example 2: Formation of Perfusable Microvessels Having Smooth and Continuous Boundaries

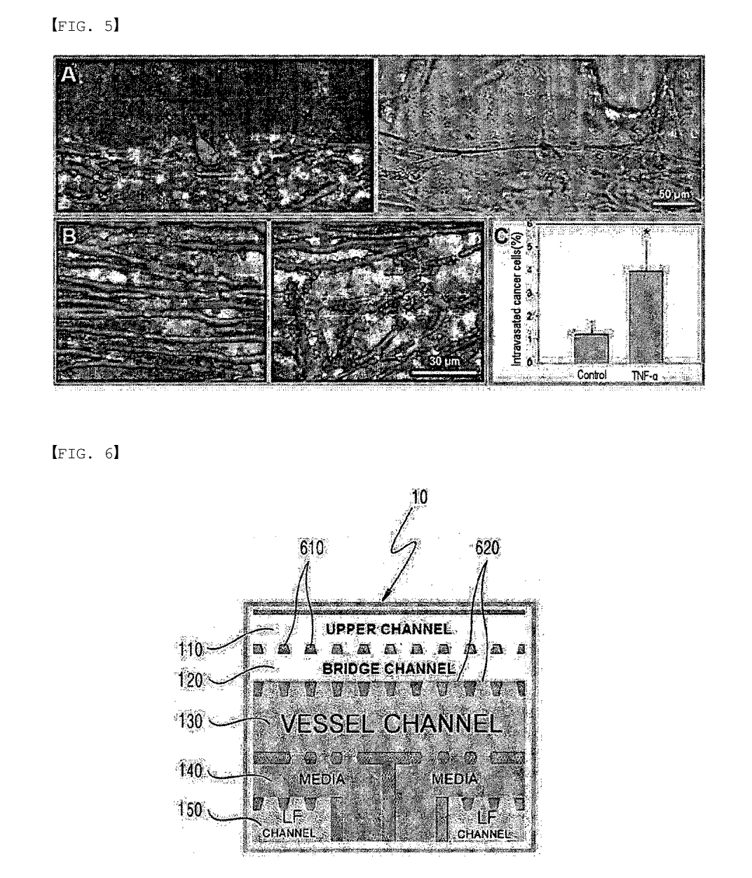

Formation of smooth and continuous vessel boundaries is important in reproducible data analysis and the use of microvessels in further studies. Thus, the present inventors have optimized the HUVEC cell concentration to form microvessels having smooth and continuous boundaries. The present inventors tested three conditions (3, 6, and 9×10⁶ HUVECs/ml and 7×106 LF/ml) using 4-5 chips per condition, and results are expressed as the mean of three independent experiments. FIG. 2A shows the microvessel morphology on day 7 under each condition. The microvessel boundary was strongly dependent on the HUVEC concentration. Microvessels formed with 3×10⁶ HUVECs/ml showed a rough and discontinuous boundary having scattered small sprouts. Microvessels formed with 9×10⁶ HUVECs/ml had a continuous boundary with fewer, scattered, small sprouts. However, the microvessels covered the majority of the area between the microposts in the upper region and exhibited a rough boundary. In contrast, microvessels formed with 6×10⁶ HUVECs/ml showed not only a continuous boundary without scattered small sprouts but also the smoothest boundary morphology with few sprouts protruding toward the upper region of the third channel 130 (blood vessel channel). FIG. 2B shows the success rate for microvessel formation for different HUVEC concentrations. Using 6×10⁶ HUVECs/ml resulted in the highest success rate (>77%) in terms of obtaining smooth and continuous boundary morphology. Therefore, the microvessels formed using 6×10⁶ HUVECs/ml showed the most pertinent boundary morphology according to the present invention, and this condition was used in all further experiments. FIG. 2C shows a series of micrographs during microvessel formation in the third channel 130. The HUVECs began to exhibit an elongated morphology with the small vacuoles 2 days after the seeding. At this time, the microvessels were not fully connected and no patent lumen was observed. HUVEC sprouts began to form a continuous lumen on day 4, and showed an elongated shape. Then, the HUVEC sprouts began to merge to form a microvessel in the third channel 130 (vessel channel). Furthermore, HUVECs in the upper region between the microposts 610 began to migrate toward the lower direction, forming a flat and smooth microvessel boundary. The HUVEC sprouts began to be fused to yield a fully lumenized microvessel on day 7, and HUVECs in the upper region between the microposts 610 had migrated lower compared to those on day 4 to form the smooth microvessel boundary. The microvessel maintained its morphological characteristics for 14 days or more.

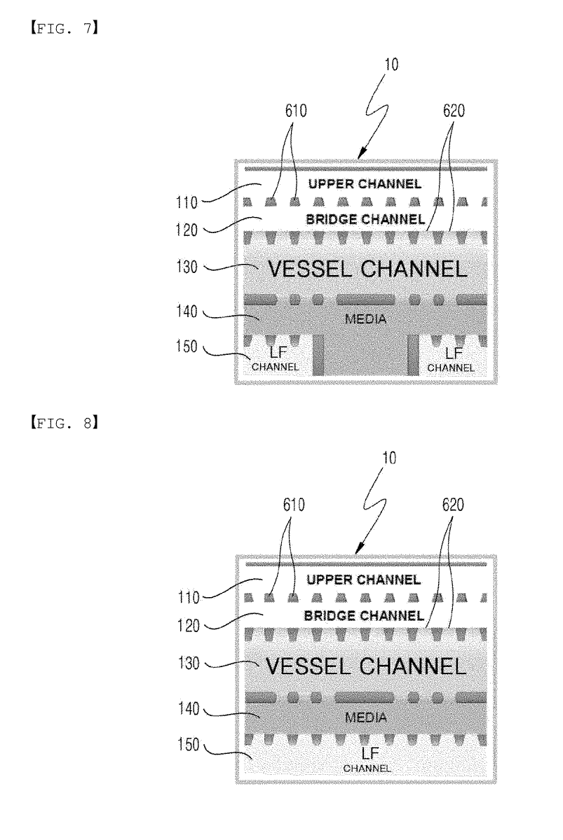

After formation of the microvessel (days 7-8), nuclei (blue) and CD31 (red) were immunostained and imaged with a confocal microscope (FIG. 3A). Cross-sectional images of the microvessel at various positions revealed patent three-dimensional lumens. The microvessel was composed of a clear lumen that connected both openings toward the fourth channel 140 (medium channel). The present inventors introduced microbeads (Sigma) through the microvessels, and as a result, confirmed that about 94% (101 of 108 chips) of the microvessels opened toward the medium channel and the medium could be perfused through the vascular lumens.

The present inventors introduced 20 kDa FITC-dextran solution into the microvessel to verify the patency of the lumen, and visualized the behavior of the lumen using fluorescence microscopy. The FITC-dextran filled the microvessel lumen without severe focal leakage through the wall (FIG. 3B). The permeability coefficient of the microvessel was 1.58±0.32×10⁻⁶ cm/s (mean±standard error, n=5) as determined by a time-lapse acquisition of the FITC-dextran intensity profile. The permeability coefficient was in the range of those for other in vitro models of blood vessels, indicating that the microvessel includes appropriate cell-cell junctions. The present inventors analyzed the junctions by fluorescent immunostaining for claudin-5 and ZO-1 (FIGS. 3C and 3D). The fluorescence images of two cell-cell junctions showed a smooth, clear elongated morphology without any disrupted or wrinkled positions, which are the basic characteristics of cell-cell junctions in normal blood vessels in vivo.

### Example 3: Cancer Angiogenesis Assay

The present inventors analyzed the angiogenic potential of cancer cells by using the microvessels having a smooth vessel wall. After formation of the microvessels using the microfluidic chip 10 according to Example 2 (day 7-8), the present inventors introduced U87MG, a glioblastoma cell line having high angiogenic potential, and a fibrin mixture into the first channel 110 (upper channel), while the second channel 120 (bridge channel) was filled with fibrin gel only. The microvessel was regarded as pre-existing at the cancer site, whereas the U87MG cells in the perivascular region secrete angiogenic factors to induce production of angiogenic sprouts toward the microvessels. Three different concentrations (control 0/ml, 2.5×10⁶ and 5×10⁶) of U87MG cells with or without bevacizumab (bev) were used, and an anti-VEGF antibody used widely for targeting angiogenesis was used. The angiogenic sprouting from the microvessels was analyzed, and the mean values of six chips per condition were calculated.

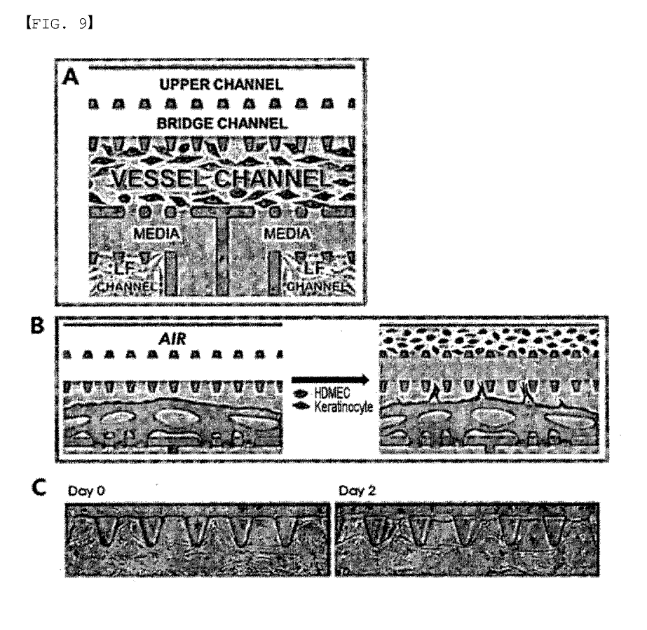

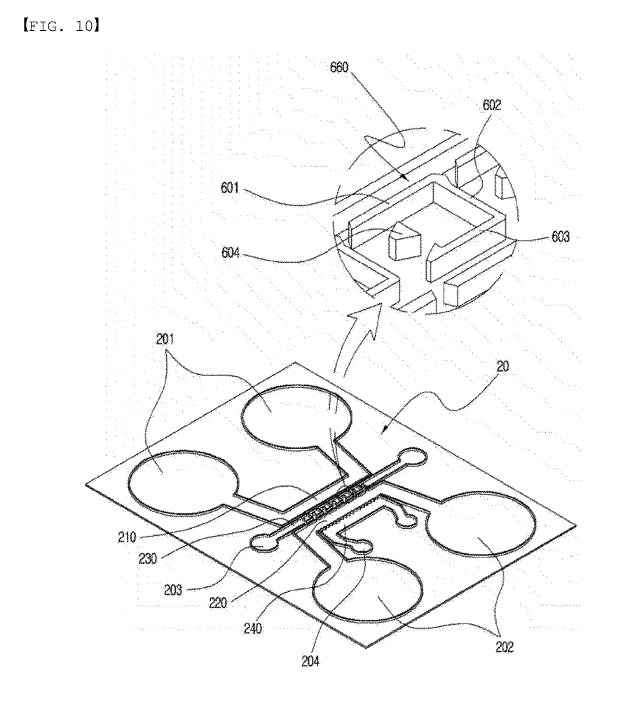

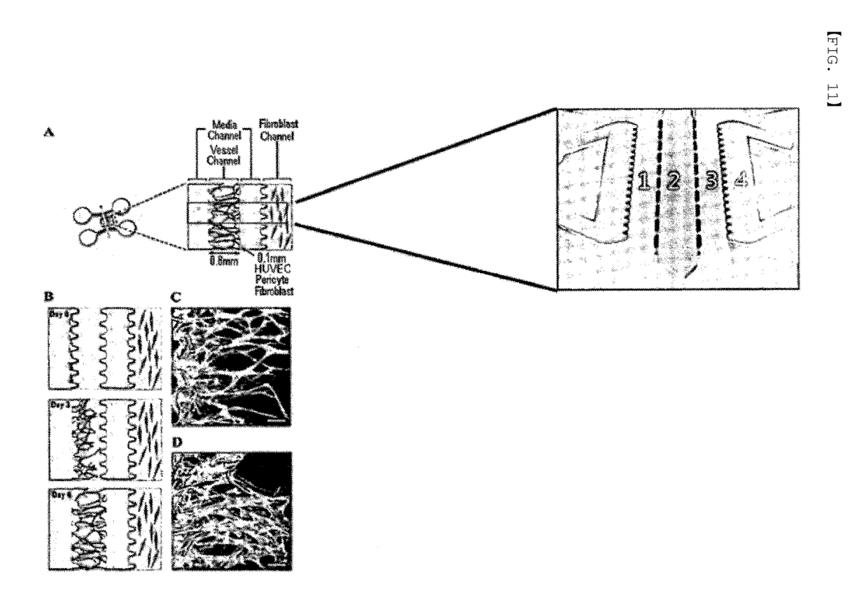

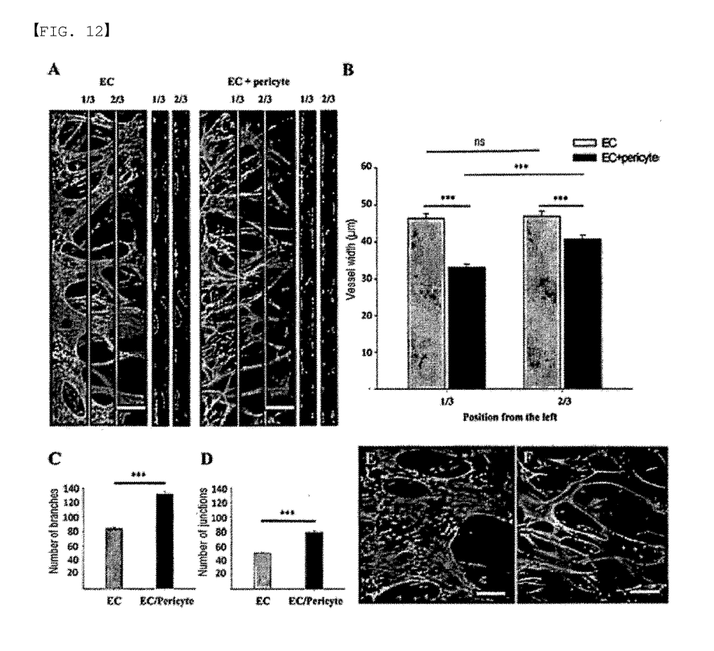

Three days after introduction of U87MG cells into the chip, the microvessels were imaged, and the number and coverage area of angiogenic sprouts under each condition were quantified. As shown in FIG. 4A, most of the microvessels without U87MG cells showed few angiogenic sprouts, but the microvessels with U87MG cells showed a considerable number of angiogenic sprouts. Furthermore, most microvessels treated with bevacizumab had a flat boundary without angiogenic sprouts. In some bevacizumab-treated samples, sprout regression which existed prior to introduction of cancer cells and bevacizumab was observed (FIG. 4A). It was postulated that this phenomenon was due to the anti-angiogenic effect of bevacizumab.

Next, the number and coverage area of sprouts. The microvessels with cancer cells exhibited significantly increased sprout numbers and coverage areas compared to the control (FIGS. 4B and 4C). Furthermore, bevacizumab treatment attenuated the angiogenic potential of the U87MG cells, drastically decreasing the number and coverage area of the microvessel sprouts. These trends of the sprout induction by cancer cells and sprout attenuation by anti-VEGF treatment are in agreement with previous in vivo reports, and demonstrate that this microvessel model is appropriate for the assessment of drugs targeting cancer angiogenesis.

### Example 4: Cancer Intravasation Assay

As a vessel to assess the transendothelial migration of MDA-MB-231 cancer cells, the microvessel obtained in the present invention was used. The cells are conventionally used for the assessment of transendothelial migration and have aggressive metastatic potential. As shown in FIG. 1D, cancer cells were attached to the interface between the fibrin gel and the second channel (bridge channel), and were incubated for 2-3 days to allow them to migrate toward and penetrate into the microvessel walls. FIG. 5A shows fluorescence and differential interference contrast (DIC) micrographs of a MDA-MB-231 cancer cell (green) in the process of intravasation through the microvessel wall (red). The number of cancer cells intravasated into the microvessels was manually counted. In this Example, the microvessels were treated with 5 ng/ml TNF-α 24 hours before the introduction of the cancer cells, and the rate of cancer intravasation was compared with that of the control. The adheren junction VE-cadherin in the TNF-α treated microvessels exhibited disrupted continuity and wrinkled morphology compared to control microvessels (FIG. 5B). The permeability of TNF-α-treated microvessels over 24 h was 2.22±0.67×10⁻⁶ cm/s (mean±standard error, n=5 chips), which was 1.4-fold higher than that under the normal condition. These results confirm the effect of TNF-α on junctional disruption of endothelial cells, and are in agreement with previous reports. In addition, the effect of TNF-α on cancer cell intravasation was analyzed. the rate of intravasation with or without TNF-α treatment was compared (n=7 chips per condition). The rate of cancer intravasation of TNF-α-treated microvessels was 3-fold higher than that of the control (FIG. 5C). This result suggests that TNF-α treatment exerted marked effects on cancer cell intravasation, in agreement with previous reports.

### Example 5: Blood Vessel-Pericyte Co-Culture

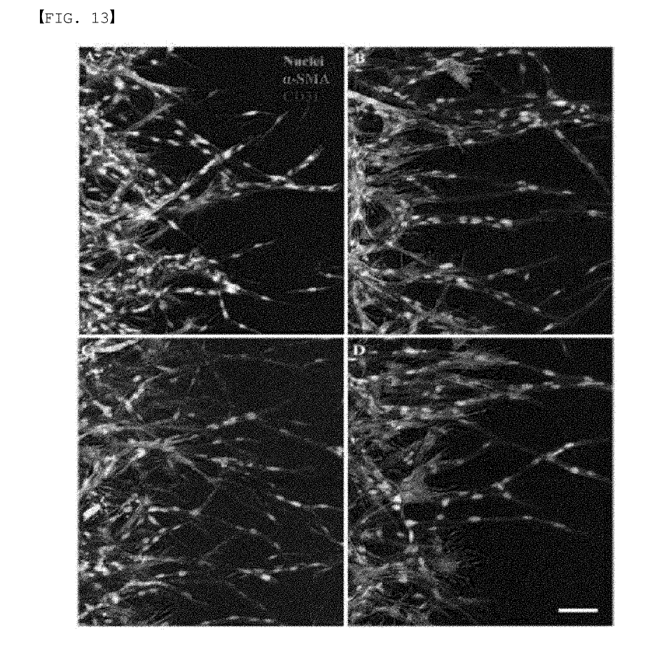

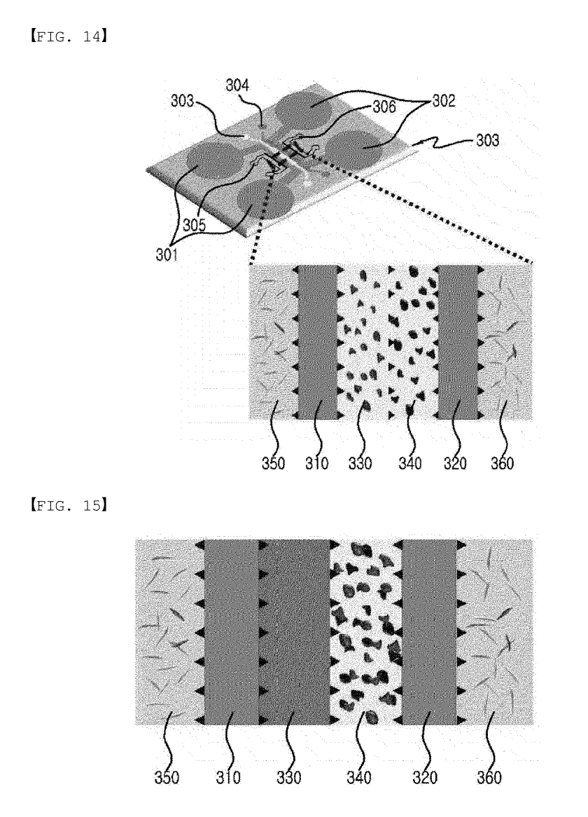

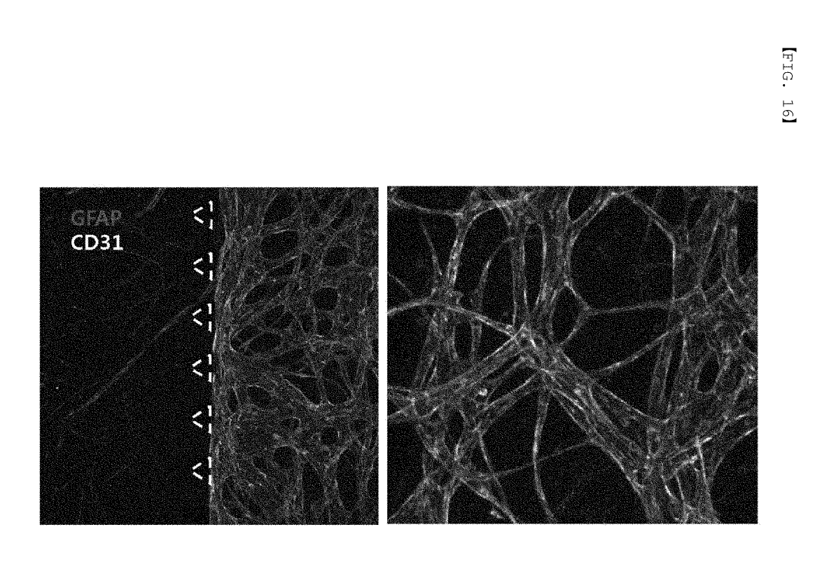

Using the microfluidic chip 20 (FIG. 11A) of the present invention, blood vessels and pericytes were co-cultured. 2.5 mg/ml fibrin gel was patterned on the third channel 230 of the microfluidic chip 20, and a mixture of dermal fibroblasts and fibrin gel was patterned on the fourth channel 240 at a concentration of 5 to 10 million/ml, thereby forming a three-dimensional environment. HUVECs (blood vessel cells) and pericytes (pericytes were obtained from Procell, Germany) were mixed at a ratio of 5:1, and a cell suspension having a cell concentration of a total of 6 million/ml was obtained and injected into the first channel 210. Next, the chip device was tilted by an angle of 90° for 30 minutes such that the cells were attached to the fibrin gel at the interface between the first channel 210 and the third channel 230. Skin fibroblasts injected into the fourth channel 240 formed an asymmetric gradient of growth factor between the first channel 210 to the third channel 230, and thus the attached vascular cells showed characteristic collective migration similar to in vivo angiogenesis within 6 days. At this time, pericytes were also formed while surrounding the blood vessels. As the fibroblasts injected into the fourth channel 240 and formed the growth factor gradient, dermal fibroblasts were used. In this case, it was confirmed that the contact between the blood vessels and the pericytes became closer to each other. The morphological characteristics of the blood vessels formed in the microfluidic chip according to this Example were analyzed, and as a result, it was shown that (a) when blood vessels were co-cultured with pericytes, the vessels became much thinner and the number of vascular branches increased, compared to when the blood vessels were cultured alone. This suggests that the pericytes have the effect of inhibiting the expansion of blood vessels by interaction with the blood vessels. Fluorescence micrographs were analyzed by a computer, thereby quantifying (b) the width of blood vessel, (c) the number of blood vessel branches; and (d) the number of junctions. As a result, it was confirmed statistically and quantitatively that when blood vessels were co-cultured with pericytes, the width of the blood vessels decreased and the number of branches and junctions increased. These results are shown in FIGS. 12A to 12F.

### Example 6: Mimicking of Skin Immune System—Observation of the Response of Blood Vessels and Pericytes to Immune Cells

FIG. 13 shows the results obtained by adding VEGF-A (b), TNF-a (c) and IL-1a (d), which are typical factors that frequently occur in the peritumoral environment and inflammatory conditions, to the blood vessel-pericytes co-cultured in Example 5 above, and observing the responses of the blood vessels and the pericytes to these factors in comparison with a control (a). In FIG. 13, light pink indicates nuclei, dark pink indicates CD31 that stains the blood vessels, and green indicates α-SMA that stains the pericytes. In the test groups (c and d) mimicking the situation where the immune system was activated, it can be observed that the pericytes sprouted out from the blood vessels and formed many filopodia, compared to the control group. This mimics the situation where blood vessels become unstable and angiogenesis increases.

### Example 7: Blood Vessel-Astrocyte Co-Culture

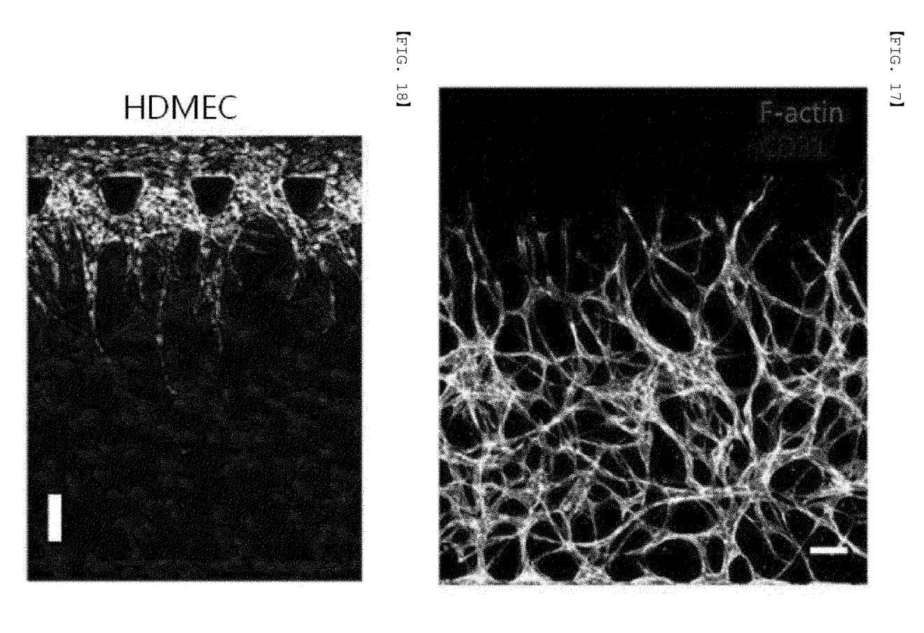

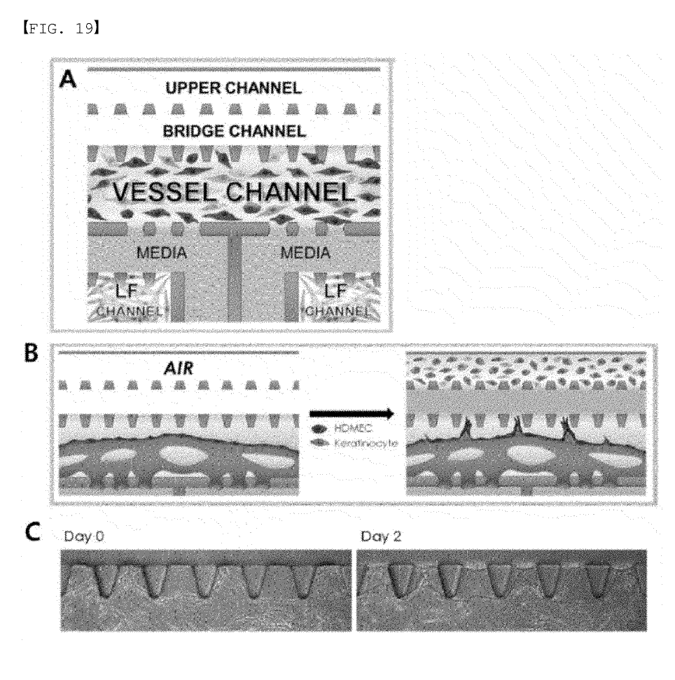

For a microfluidic chip 30 (FIGS. 14 and 15), two hydrogels were patterned adjacent to each other (third channel 330 and fourth channel 340), unlike a conventional chip. In order to perform patterning such that air bubbles do not enter the hydrogel channel formed in the third channel 330 and the fourth channel 340, hydrogel was filled in the first channel 340 and cured for 2-5 minutes, and then the third channel 330 was filled with hydrogel. On the fifth channel 350 and the sixth channel 360, fibrin hydrogel mixed with 5-10 million/ml of lung fibroblasts (Lonza, 3D ECM; obtained from Sigma Aldrich Korea) was patterned. Medium was added to the first channel 310 and the second channel 320. To see the effect of the physical junction of astrocytes to blood vessel cells, 5 million/ml of blood vessel cells-astrocytes were added to each of the third channel 330 and the fourth channel 340, which were divided so as to be close to move toward each other but away from each other. FIGS. 16A and 16B are confocal micrographs showing the results. When the blood vessel cells and the astrocytes were cultured separately in the third channel 330 and the fourth channel 340, angiogenesis in the channel containing the astrocytes was inhibited (FIG. 16A). In addition, blood vessel cells and astrocytes were mixed at a ratio of 1:1 and co-cultured in the third channel 330 or the fourth channel 340. In this case, the mixture of the two types of cells was added at a concentration of 10 million/ml. When the blood vessel cells and the astrocytes were co-cultured in the same channel, it was observed that the endfoot of the astrocytes was formed toward the blood vessels (FIG. 16B). In FIG. 16B, green fluorescence indicates GFAP that selectively stains the astrocytes, and white indicates CD31 that stains the blood vessel cells. FIGS. 14 and 15 show the structure of the microfluidic chip used in this Example.

In addition, two fibrin hydrogels were patterned adjacent to each other, and blood vessel cells or lymphatic vessel cells were cultured alone in the fourth channel 340, and in vivo blood vessel/lymphatic vessel formation processes were observed. Over 2-3 days, a blood vessel or lymphatic vessel network was first formed in the fourth channel 340, and then over 2-3 days, angiogenesis could be induced from the generated blood vessel network. This confirms that it is possible to sequentially and accurately mimic in vivo blood vessel formation processes in which a blood vessel network is formed through angiogenesis following vasculogenesis. In addition, it is also possible to mimic the process of lymphatic vessel formation and reproduce lymphatic vessels in this context. The results are shown in FIG. 17. In FIG. 17A, green fluorescence indicates F-actin, and red indicates CD31 that selectively stains blood vessels. It is a confocal micrograph showing an intermediate process in which angiogenesis was generated in the left empty half of the hydrogen after angiogenesis in the right half of the hydrogel was completed. FIG. 17B is a micrograph of lymphatic vessel cells which were grown and stained in the same manner. In FIG. 17B, green indicates Podoplanin known to stain only lymphatic vessels, and red indicates F-actin.

### Example 8: Horizontal Culture of Skin's Structural Layers

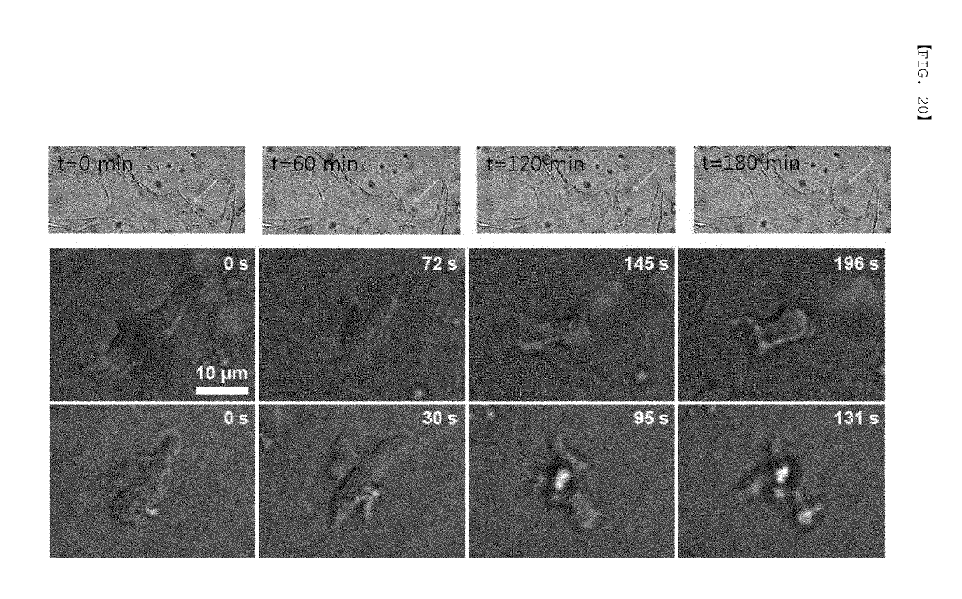

In this Example, the skin's structural layer was cultured horizontally. In this Example, the microfluidic chip 20 having the structure shown in FIG. 11A was used. The height of the chip was adjusted to 100 μm, and the height of each channel was adjusted to 500 μm to 800 μm, and triangular or hexagonal posts were disposed between the channels. These posts could surface tension, and thus hydrogel could be patterned sequentially on the third channel 230 and the fourth channel 240 without mixing. On the third channel 230, 2.5 mg/ml fibrin gel mixed with 2 million/ml of dermal fibroblasts was patterned, and on the fourth channel 240, fibrin gel mixed with 5-10 million/ml of keratinocytes was patterned. After the concentration of the cell supernatant was adjusted such that HDMECs (blood vessel cells) had a cell concentration of 7 million cells/ml, the cell supernatant was injected into the first channel 210, and the chip was tilted by an angle of 90° for 30 minutes such that the cells were attached to the fibrin gel at the interface between the first channel 210 and the third channel 230 by gravity. Next, EGM2-MV (HDMEC culture medium) was added to the first channel 210, and Epilife (keratinocyte culture medium, Thermo Fisher Scientific) was injected into the second channel 220, followed by culture of the cells. The keratinocytes injected into the fourth channel 240 formed an asymmetric concentration gradient of growth factor between the first channel 210 to the third channel 230, and thus the attached blood vessel cells exhibited characteristic collective migration similar to in vivo angiogenesis within about 6 days. In this case, the dermal fibroblasts in the third channel 230 helped the growth of blood vessels. FIG. 18 is a confocal micrograph showing the results of observation. In FIG. 18, the green fluorescence (HDMEC marker CD31) portion on the left, in which the blood vessel cells sprout out, is the interface between the first channel 210 and the third channel 230. The blue points in the third channel 230 are the nuclei of the dermal fibroblasts. In this Example, the first layer 210 mimicked the skin's subcutaneous fat layer having blood vessels; the third channel 230 mimicked the dermal layer having blood vessels and fibrous cells; and the fourth channel 240 mimicked the epidermal layer having keratinocytes.

### Example 9: Mimicking of Immune Function of Skin's Structural Layers

In this Example, the immune function of the skin's structural layers was mimicked. In this Example, the microfluidic chip 10 having the structure shown in FIG. 19A was used. The height of the chip device was adjusted to 100 μm, and the height of each channel was adjusted to 500-800 μm. Triangular or hexagonal posts were disposed between the channels, and thus hydrogel could be patterned sequentially on the first channel 110, the third channel 130 and the fifth channel 150 without mixing. 2.5 mg/ml fibrin gel mixed with 5-10 million/ml of endothelial cells was patterned on the third channel 130, and fibrin gel mixed with 5-10 million/ml of angiogenic cells (fibroblasts, msc, cancer, etc.) was patterned on the fifth channel 151 or 152. When blood vessel culture medium was injected into the fourth channel 140, the angiogenic cells injected into the fifth channel 151 or 152 formed an asymmetric concentration gradient of growth factor between the third channel 130 and the fourth channel 140, and thus the blood vessel cells formed a perfusable blood vessel that opened only toward the fourth channel 140, without about 6 days. Next, 2.5 mg/ml fibrin gel mixed with 5-10 million/ml of keratinocytes was patterned on the first channel 110, and the keratinocyte culture medium Epilife was injected into the second channel 120. In the third channel 130, a blood vessel network that opened on one side formed new blood vessels by the influence of the keratinocyte growth factor present in the first channel 110. Next, immune cells were injected into the fourth channel 140, and then the immune cells entered the blood vessels through the opened portion, and then extravasated from the blood vessel walls, and were positioned between the second channel 120 and the third channel 130. Thus, this Example suggested a platform capable of observing in vivo blood vessels and tissue structures, and the migration and response of immune cells therein (see FIG. 20). It reproduced the process in which cancer cells and immune cells extravasate through blood cells, thereby mimicking the process of formation of the skin immune system, for example, the migration of progenitor cells of the skin immune system through blood vessel walls and the differentiation of the progenitor cells in skin tissue. Thus, it was confirmed that the present invention makes it possible to mimic the skin immune system in vitro more efficiently than when mature immune cells are used.

### DESCRIPTION OF REFERENCE NUMERALS

10, 20, 30: microfluidic chip;
110, 210, 310: first channel;
120, 220, 320: second channel;
130: third channel; 140: fourth channel;
150, 151, 152: fifth channel;
160: barrier;
201, 301: first medium reservoir;
202, 302: second medium reservoir;
203: blood vessel channel inlet;
204: cell channel inlet;
230: blood vessel channel; 240: cell channel;
303: first blood vessel channel inlet;
304: second blood vessel channel inlet;
305: first cell channel inlet;
306: second cell channel inlet;
330: first blood vessel channel;
340: second blood vessel channel;
350: first cell channel;
360: second cell channel;
610: microstructures;
620: gap;
660: barrier structures.

1. A biological tissue chip configured such that blood vessels or lymphatic vessels and cells, co-cultured in vitro, interact with each other, the biological tissue chip comprising:
at least one blood vessel channel and blood vessels or lymphatic vessels or a combination of blood vessels or lymphatic vessels, formed in the blood vessel channel;
at least one cell channel and cells cultured in the cell channel; and
at least one medium channel,
wherein the blood vessel channel, the cell channel and the medium channel are disposed adjacent and parallel to one another such that they are in fluidic communication with one another;
both sides or one side of the blood vessel channel is adjacent to the medium channel, both sides or one side of the cell channel is adjacent to the other side of the medium channel, and two or more barrier structures or microstructures are disposed at an interface between adjacent two of the channels with a gap;
the medium channel is connected with a medium reservoir such that they are in fluidic communication with each other, and each of the blood vessel channel and the cell channel is connected with its inlet such that they are in fluidic communication with each other;
each of the channels allows an interaction between biochemical substances contained in the channels through the gap;
blood vessels or lymphatic vessels are formed from angiogenic or lymphangiogenic cells in the blood vessel channel, and cells are cultured in the cell channel; and
the cultured cells interact with the formed blood vessels or lymphatic vessels.

2. The biological tissue chip of claim 1, wherein the biological tissue is a skin tissue comprising a subcutaneous fat layer, a dermal layer and a horny layer.

3. The biological tissue chip of claim 1 or 2, wherein the cells are one or more selected from the group consisting of pericytes, astrocytes, cancer cells, immune cells, glial cells, mesothelial cells, fibroblasts, smooth muscle cells, pericytes, neuroglial cells, stem cells, stem cell-derived cells, and cells that interact with vascular endothelium.

4. The biological tissue chip of claim 3, wherein the co-cultured cells are mutated cells, transfected cells, or mutated and transfected cells.

5. The biological tissue chip of claim 1 or 2, wherein the angiogenic or lymphangiogenic cells are one or more selected from the group consisting of endothelial cells, epithelial cells, cancer cells, stem cells, stem cell-derived cells, and vascular endothelial progenitor cells.

6. The biological tissue chip of claim 5, wherein the angiogenic or lymphangiogenic cells are mutated cells, transfected cells, or mutated and transfected cells.

7. The biological tissue chip of claim 1, wherein a third channel 130 as the blood vessel channel, a first channel 110 and a fourth channel 140 as the medium channel, and a fifth channel 150 as the cell channel are disposed parallel to one another,
wherein:
one side of the first channel 110 is adjacent to one side of the second channel 120; the other side of the second channel 120 is adjacent to one side of the third channel 130; the other side of the third channel 130 is adjacent to one side of the fourth channel 140; the other side of the fourth channel is divided into two or more chambers by a barrier extending perpendicular to the other side, and each of the chambers includes a fifth channel 151 or 152 connected to one side of the fourth channel so as to be in fluidic communication with the fourth channel.

8. The biological tissue chip of claim 1, wherein
the medium channel comprises: a first channel 210 configured to be in fluidic communication with a first medium reservoir 201; and a second channel 220 configured to be in fluidic communication with a second medium reservoir 202 and disposed parallel to the first channel 210;
the blood vessel channel comprises: a third channel 230 configured to be in fluidic communication with a blood vessel channel inlet 203 and disposed between the first channel 210 and the second channel 220 and disposed parallel to one side of each of the first channel 210 and the second channel 220; and
the cell channel comprises a fourth channel 240 configured to be in fluidic communication with a cell channel inlet 204 and adjacent to the other side of the second channel 220 and disposed parallel to the second channel 220.

9. The biological tissue chip of claim 1, wherein
the medium channel comprises: a first channel 310 configured to be in fluidic communication with a first medium reservoir 301; and a second channel 320 configured to be in fluidic communication with a second medium reservoir 302 and disposed parallel to the first channel 310;
the blood vessel channel comprises: a first blood vessel channel 330 configured to be in fluidic communication with a first blood vessel channel inlet 303 and adjacent to one side of the first channel 310; and a second blood vessel channel 340 configured to be in fluidic communication with a second blood vessel channel inlet 304 and adjacent to the other side of the second channel 330; and
the cell channel comprises: a first cell channel 350 configured to be in fluidic communication with a first cell channel inlet 305 and adjacent to the other side of the first channel 310 and disposed parallel to the first channel 310; and a second cell channel 360 configured to be in fluidic communication with a second cell channel inlet 306 and adjacent to the other side of the second channel 320 and disposed parallel to the second channel 320.

10. The biological tissue chip of claim 7,
wherein endothelial cells and fibrin gel are patterned on the third channel 130, angiogenic cells and fibrin gel are patterned on the fifth channel 151, 152, vascular endothelial cell culture medium is injected into the fourth channel 140, keratinocytes and fibrin gel are patterned on the first channel 110, keratinocyte culture medium is injected into the second channel 120, and the cells are cultured, whereby the endothelial cells in the third channel 130 form perfusable blood vessels opened only toward the fourth channel 140, and form new blood vessels toward the first channel.

11. The biological tissue chip of claim 8, wherein dermal fibroblasts and fibrin gel are patterned on the third channel 230, keratinocytes and fibrin gel are patterned on the fourth channel 240, vascular endothelial cells are injected into the first channel 210 and attached to the interface between the first channel 210 and the third channel 230, endothelial cell culture medium is injected into the first channel 210, and keratinocyte culture medium is injected into the second channel 220, whereby the attached endothelial cells form new blood vessels toward the fourth channel 240.

12. The biological tissue chip of claim 8, wherein fibrin gel is patterned on the third channel 230, dermal fibroblasts and fibrin gel are patterned on the fourth channel, vascular endothelial cells and pericytes are injected into the first channel 210, and these cells are attached to the interface between the first channel 210 and the third channel 230 and cultured, whereby the attached vascular endothelial cells form new blood vessels toward the fourth channel 240.

13. The biological tissue chip of claim 9, wherein fibroblasts and fibrin gel are patterned on the fifth channel 350 and the sixth channel 360, medium is injected into the first channel 310 and the second channel 320, angiogenic cells and fibrin gel are patterned on the fourth channel 340, and then astrocytes and fibrin gel are patterned on the third channel 330, followed by culture.

14. The biological tissue chip of claim 9, wherein fibroblasts and fibrin gel are patterned on the fifth channel 350 and the sixth channel 360, medium is injected into the first channel 310 and the second channel 320, and a mixture of angiogenic cells and astrocytes together with fibrin gel are patterned on the fourth channel 340, followed by culture.

15. A method of forming microvessels in vitro in the biological tissue chip of claim 7, the method comprising:
(i) adding a mixture of fibroblasts and fibrin to the fifth channel, and a mixture of vascular endothelial cells and fibrin to the third channel, followed by culture; and
(ii) maintaining the first channel and the second channel in an empty state during the culture.

16. The method of claim 15, wherein a concentration of the endothelial cells is 4×10⁶ to 8×10⁶ cells/ml.

17. A method of generating cancer angiogenesis in vitro in the biological tissue chip of claim 7, the method comprising:
(i) adding a mixture of fibroblasts and fibrin to the fifth channel, and adding a mixture of vascular endothelial cells and fibrin to the third channel, followed by culture;
(ii) maintaining the first channel and the second channel in an empty channel state during the culture, thereby forming microvessels; and
(iii) injecting an angiogenic cell line into the first channel, and injecting fibrin into the second channel, followed by culture.

18. The method of claim 17, wherein the fibroblasts are lung fibroblasts (LF), the endothelial cells are HUVEC, and the angiogenic cell line is U87MG cell line (ATCC HTB-14™).

19. A method of generating cancer intravasation in vitro in the biological tissue chip of claim 7, the method comprising:
(i) adding a mixture of fibroblasts and fibrin to the fifth channel, and adding a mixture of vascular endothelial cells and fibrin gel to the third channel, followed by culture;
(ii) maintaining the first channel and the second channel in an empty state during the culture, thereby forming microvessels;
(iii) injecting an angiogenic cell line into the first channel, and injecting fibrin gel into the second channel, followed by culture, thereby generating cancer angiogenesis;
(iv) attaching cancer cells to the fibrin gel of the second channel;
(v) supplying a medium for cancer cell growth to the first channel, and then adding growth factor-free medium to the first channel.

20. A method of screening an anticancer drug candidate in vitro in the biological tissue chip of claim 7, the method comprising:
(i) adding a mixture of fibroblasts and fibrin to the fifth channel, and adding a mixture of vascular endothelial cells and fibrin to the third channel, followed by cultured;
(ii) maintaining the first channel and the second channel in an empty channel state during the culture, thereby forming microvessels;
(iii) injecting an angiogenic cell line and a sample to be analyzed into the first channel, and injecting fibrin into the second channel, followed by culture; and
(iv) determining that the sample is an anticancer drug candidate, when cancer angiogenesis is not generated.

21. A method for generating blood vessels or lymphatic vessels and cells, which interact with each other in vitro, the method comprising:
sequentially or simultaneously injecting one or more, selected from the group consisting of angiogenic cells, lymphangiogenic cells, extracellular matrices, cell culture media, angiogenic factors, lymphangiogenic factors and co-culture cells, into one or more independent channels of the biological tissue chip according to claim 1;
culturing angiogenic cells;
inducing blood vessel formation; and
culturing co-culture cells.

22. A method for generating blood vessels or lymphatic vessels and cells, which interact with each other in vitro, the method comprising the steps of:
(a) injecting extracellular matrix and angiogenic or lymphangiogenic cells into the blood vessel channel of the biological tissue chip according to claim 1;
(b) injecting extracellular matrix or a combination of extracellular matrix and co-culture cells into the cell channel; and
(c) injecting cell culture medium, angiogenic or lymphangiogenic factor, or a combination of cell culture medium and angiogenic or lymphangiogenic factor into the medium channel, inducing blood vessel or lymphatic vessel formation in the blood vessel channel, and culturing the co-culture cells in the cell channel.

23. A method for generating blood vessels or lymphatic vessels and cells, which interact with each other in vitro, the method comprising the steps of:
(a) injecting extracellular matrix or a combination of extracellular matrix and co-culture cells into the blood vessel channel of the biological tissue chip according to claim 1, and forming a cell adhesion surface for cell adhesion at an interface between the blood vessel channel and the medium channel;
(b) injecting angiogenic cells into the medium channel, and attaching the angiogenic cells to the cell adhesion surface;
(c) injecting extracellular matrix or a combination of extracellular matrix and co-culture cells into the cell channel; and
(d) injecting cell culture medium, angiogenic factor, or a combination of cell culture medium and angiogenic factor into the medium channel, culturing in the angiogenic cells in the blood vessel channel, and inducing blood vessel formation.

24. The method of any one of claims 21 to 23, wherein the angiogenic cells are one or more selected from the group consisting of endothelial cells, epithelial cells, cancer cells, stem cells, stem cell-derived cells, and endothelial progenitor cells.

25. The method of claim 24, wherein the angiogenic cells are mutated cells, transfected cells, or mutated and transfected cells.

26. The method of any one of claims 21 to 23, wherein the extracellular matrix is one or more selected from then group consisting of collagen gel, fibrin gel, Matrigel, self-assembled peptide gel, polyethylene glycol gel, and alginate gel.

27. The method of any one of claims 21 to 23, wherein the co-culture cells are one or more selected from the group consisting of astrocytes, glial cells, mesothelial cells, fibroblasts, smooth muscle cells, pericytes, neuroglial cells, stem cells, stem cell-derived cells, and cells that interact with vascular endothelium.

28. The method of claim 27, wherein the co-culture cells are mutated cells, transfected cells, or mutated and transfected cells.

29. The method of any one of claims 21 to 23, wherein the extracellular matrix or the cell culture medium comprises one or more selected from the group consisting of drugs, soluble factors, insoluble factors, biomolecules, proteins, nanomaterials, and siRNA.

30. A biological tissue chip of mimicking a skin immune system in vitro, the chip comprising immune cells co-cultured in a cell channel of a cell tissue chip set forth in claim 1 or 2.
